(19) United States
(12) Patent Application Publication
(10) Pub. No.: US 2020/0095549 A1
(43) Pub. Date: Mar. 26, 2020

(54) METHOD FOR INDUCING REGULATORY T CELL

(71) Applicant: KYOTO UNIVERSITY, Kyoto (JP)

(72) Inventors: Shimon SAKAGUCHI, Kyoto (JP); Yohko KITAGAWA, Kyoto (JP); Norihisa MIKAMI, Kyoto (JP)

(21) Appl. No.: 16/470,353

(22) PCT Filed: Dec. 19, 2017
(86) PCT No.: PCT/JP2017/045493

§ 371 Date: Dec. 4, 2019

(30) Foreign Application Priority Data
Dec. 19, 2016 (JP) 2016-245436

(51) Int. Cl. C12N 5/0783; G01N 33/50; A61K 35/17; A61P 37/02

(52) U.S. Cl. CPC C12N 5/0637; G01N 33/505; C12N 2510/00; A61P 37/02; A61K 35/17

(57) ABSTRACT

Provided is a method for inducing regulatory T cells, comprising a step of inhibiting the expression of Satb1 or the function of Satb1 in peripheral conventional T cells.

### ART RELATED

The present application relates to a method for inducing regulatory T cells from peripheral T cells.

### BACKGROUND ART

CD25-positive CD4-positive regulatory T cells that are inherent in the immune system specifically express a transcriptional factor Foxp3. Deletion or mutation of Foxp3 may impair the production or development and also the immunosuppressive function of regulatory T cells. On the basis of experimental results wherein normal T cells exerted the immunosuppressive function when Foxp3 was ectopically expressed therein, Foxp3 gene has been regarded as a master gene for the generation and the function of regulatory T cells. Mutations in Foxp3 gene in human been to impair the production of regulatory T cells in most cases, resulting in abnormal regulation of immune responses to self and non-self antigens.

Recently, it has been reported that Foxp3 expressing CD8-positive cells play an important role in the control of immune responses.

Induced regulatory T cells having functions and phenotype similar to those of endogenous regulatory T cells can be experimentally generated by inducing Foxp3 expression in mouse CD4-positive T cells by stimulating the cells with an antigen in the presence of TGFβ. The expression of Foxp3 in the induced regulatory T cells is, however, unstable and the cells cannot maintain the immune-suppressing function stably, and hence it is difficult to employ the induced regulatory T cells in clinical applications.

Recent studies have shown that the instability of Foxp3 expression in the induced regulatory T cells is primarily due to differences in epigenetic regulation of Foxp3 gene. Epigenetics is defined as changes in gene expression or phenotype that are inherited during cell division without changes in DNA sequence. Epigenetic control of gene expression refers to gene expression control performed by modification of DNA or chromatin without altering the DNA sequence. In recent years, it has become clear that regulation of gene expression specific to cell lineages depends on control by transcription factors and epigenetic control. Epigenetic control in regulatory T cells involves the specific demethylation of regulatory regions of genes encoding various functional proteins specific for regulatory T cells, including Foxp3. Previous findings suggest that regulatory T cells are unstable in the expression of genes specific for regulatory T cells, including Foxp3 gene, because the demethylated region specific for regulatory T cells remains methylated. By introducing epigenetic modification specific to regulatory T cells, it is desirable to develop not only stable expression of Foxp3 but also methods for producing functionally stable regulatory T cells.

Non Patent Literature 1 describes that a Foxp3 positive cell induced in vitro by using a mixture of IL-2, TGF-β, retinoic acid, rapamycin and butyric acid has immunosuppressive activity. The expression of the induced Foxp3 in this report is unstable.

Non Patent Literature 2 reports that regulatory T cells are produced in vitro from peripheral T cells using TGF-β and rapamycin. Non Patent Literature 2 proposes that graft versus host disease (GVHD) occurring in transplantation is inhibited or treated by transferring thus obtained regulatory T cells into the living body.

Non Patent Literature 3 reports that division of the T cells is inhibited by the function of regulatory T cells and apoptosis when rapamycin is continuously administered to a humanized mouse into which peripheral blood-derived T cells have been transferred. This literature also describes adverse reactions of rapamycin.

### PRIOR ART DOCUMENTS

### Non Patent Literatures

[Non Patent Literature 1] Schmidt A., Eriksson M. et al., Comparative analysis of protocols to induce human CD4⁺Foxp3⁺ Regulatory T cells by combinations of IL-2, TGF-beta, retinoic acid, rapamycin and butyrate, ProS One. 11(2): e0148474, 2016

[Non Patent Literature 2] Hippen K. L., Merkel S. C., et al., Generation and large-scale expansion of human induced regulatory T cells that suppress graft-versus-host disease, Am J Transplant. 11(6): 1148-57, 2011

[Non Patent Literature 3] Hester J., Schiopu A. et al., Low-dose rapamycin treatment increases the ability of human regulatory T cells to inhibit transplant arteriosclerosis in vivo, Am J Transplant. 12(8): 2008-2016, 2012

### SUMMARY OF INVENTION

### Problems to be Solved by Invention

An object of the present application is to provide a method for inducing stable regulatory T cells from peripheral T cells. Another object of the present application is to provide a method for inducing regulatory T cells useful for the treatment and prevention of autoimmune diseases, immune metabolic diseases, allergy, rejection occurring in organ transplantation, graft versus host disease (GVHD) and the like, and for the induction of maternal-fetal immune tolerance.

### Means to Solve the Problem

The inventors have found for the first time that the expression of Foxp3, a master gene of regulatory T cells, can be induced by inhibiting the expression of Satb1 in peripheral conventional T cells. It has been confirmed that the inhibition of Satb1 in peripheral conventional T cells induces the expression of Foxp3 thereby accelerates the differentiation into regulatory T cell, and at the same time, inhibits the differentiation into Th17 cells that produce inflammatory cytokines. On the other hand, the inhibition of Satb1 expression did not affect the differentiation into Th1 and Th2 cells. The inventors have confirmed that T cells having been expressed Foxp3 by Satb1 inhibition are likely to differentiate into stable regulatory T cells in the living body, and act as regulatory T cells to inhibit the onset of autoimmune diseases.

The present inventors also confirmed that by inhibiting the expression of Satb1 in peripheral CD8-positive cells, the expression of Foxp3 is induced in the CD8-positive cells. When cells with suppressed Satb1 expression were further cultured in the presence of a T-cell activator, Foxp3-expressing cells proliferated specifically.

As a result of these findings, the present inventors have accomplished the invention. The present application provides the following:

[1] A method for inducing or generating regulatory T cells, comprising the step of inhibiting the expression of Satb1 or the function of Satb1 in peripheral conventional T cells.
[2] The method according to [1], comprising administering a substance that inhibits the expression of Satb1 or the function of Satb1 to a subject, thereby inducing regulatory T cells in the subject.
[3] A method for inducing the expression of Foxp3 in peripheral conventional T cells in vitro, comprising the step of inhibiting expression of Satb1 in the peripheral conventional T cells.
[4] The method according to [3], further comprising the step of culturing cells with suppressed Satb1 expression in the presence of a T-cell activator
[5] The method according to any one of [1] to [4], wherein the peripheral conventional T cells are CD4-positive cells or CD8-positive cells.
[6] The method according to any one of [3] to [5], wherein the expression of Satb1 is inhibited by means of a genome editing technology.
[7] A method for inducing regulatory T cells in a subject, comprising the step of transferring Foxp3 expressing T cells obtained by the method according to any one of [3] to [6] into the subject.
[8] The method according to [2] or [7], wherein the regulatory T cells are induced for performing a treatment selected from the group consisting of the treatment of an autoimmune disease, the treatment of an immune metabolic diseases, the treatment of an allergy, the treatment of rejection occurring in organ transplantation, the treatment of a graft versus host disease occurring in organ transplantation, and the induction of maternal-fetal immune tolerance.
[9] A regulatory T cell inducer, comprising a substance that inhibits the expression of Satb1 or the function of Satb1.
[10] An agent comprising a substance that inhibits the expression of Satb1 or the function of Satb1, for use in a treatment selected from the group consisting of the treatment of an autoimmune disease, the treatment of an immune metabolic disease, the treatment of an allergy, the treatment of rejection occurring in organ transplantation, the treatment of a graft versus host disease occurring in organ transplantation, and the induction of maternal-fetal immune tolerance.
[11] A cell culture comprising T cells with suppressed Satb1 expression or suppressed Satb1 function.
[12] A method for screening a regulatory T cell inducer, comprising the steps of:

(1) treating cells expressing Satb1 with a substance that is a candidate for a regulatory T cell inducer;

(2) measuring the expression or the function of Satb1 in the cells; and

(3) selecting a substance that inhibits the expression or the function of Satb1 as a regulatory T cell inducer.

### Effects of Invention

According to the method provided herein, Foxp3 can be stably expressed in the peripheral T cells. T cells expressing Foxp3 stably differentiate into stable regulatory T cells. In other words, by the present method, stable regulatory T cells can be induced. By inducing stable regulatory T cells, it is possible to treat autoimmune diseases, immunometabolic diseases, allergies, rejection in organ transplantation, and graft versus host disease in organ transplantation, as well as to induce fetal maternal tolerance.

### BRIEF DESCRIPTION OF DRAWINGS

FIG. 1 illustrates expression levels of Stab1 in the top 100 Satb1 high-expression cells out of 293 types of human cells.

FIG. 2 illustrates results of FACS analysis on the expression of Foxp3 and CD25 in CD4-positive T cells in the thymus and spleen of a Satb1 deficient mouse (Satb1^{f1/f1}ThpokCre⁺) and a control mouse (Satb1^{f1/+}ThpokCre⁺).

FIG. 3 is a scatter plot of the genes expressed in CD4⁺CD2.5⁻ cells of a Satb1 deficient mouse (Satb1^{f1/f1}ThpokCre⁺) and a wild type mouse (Stab1^{f1/f1}).

FIG. 4 illustrates results of FACS analysis of the expression of Helios and Nrp1 in peripheral CD4⁺ T cells of a Satb1 deficient mouse (Satb1^{f1/f1}ThpokCre⁺) and a control mouse (Satb1^{f1/+}ThpokCre⁺).

FIG. 5 is a graph plotting the FACS analysis results illustrated in FIG. 4. In CD4⁺CD25⁺ T cells isolated from the peripheries of the Satb1 deficient mouse and the control mouse, the ratio of Nrp1⁺Helios⁺ Treg cells, i.e. regulatory T cells derived from thymus is shown on the left side, and the ratio of Nrp⁺Helios⁺ Treg cells, i.e. regulatory T cells derived from the periphery is shown on the right side.

FIG. 6 illustrates the demethylation degree in the CNS2 region of Foxp3 gene contained in each cell fraction isolated from the peripheries of the Satb1 deficient mouse and the control mouse. Each block corresponds to CpG residue in the amplicon.

FIG. 7 illustrates results of FACS analysis on Th1, Th2 and Th17 markers in the cells resulting from culturing peripheral CD4⁺CD25⁻CD44⁻ cells of a wild type mouse (Satb1^{f1/f1}) and a Satb1 deficient mouse (Satb1^{f1/f1}ThpokCre⁺) under conditions for inducing Th1, Th2 and Th17 cells, respectively.

FIG. 8 is a graph plotting the FACS analysis results illustrated in FIG. 7.

FIG. 9 is a diagram illustrating the outline of the experiment in which the wild type T cells (CD45.1⁺) and the Satb1 deficient mouse-derived T cells (CD45.2⁺) were mixed to be transplanted into a mouse.

FIG. 10 illustrates the results of FACS analysis of CD4⁺ T cells derived from respective animals in the recipient's mesenteric lymph nodes on day 17 after the transplantation of a mixture of the wild type T cells (CD45.1⁺) and the Satb1 deficient mouse-derived T cells (CD45.2⁺) into the recipient.

FIG. 11 illustrates the degree of demethylation in the CNS2 region of Foxp3 gene in CD4⁺ T cells derived from the respective animals in the recipient's mesenteric lymph nodes on day 17 after the transplantation.

FIG. 12 illustrates the ratios of fully demethylated amplicons in the CNS2 region of Foxp3 gene in CD4⁺ T cells derived from the respective animals obtained from the recipient mesenteric lymph nodes on day 0 and day 17 after the transplantation.

FIG. 13 illustrates change in the body weight caused when CD4⁺CD25⁻CD45RB^{hi} T cells isolated from the periphery of a Satb1 deficient mouse (Satb1^{f1/f1}ThpokCre⁺) or a wild type mouse (Satb1^{f1/f1}) were transplanted into enteritis model mice.

FIG. 14 illustrates photographs of colons of enteritis model mice on day 53 after the transplantation of CD4⁺CD25⁻CD45RB^{hi} T cells isolated from the periphery of the Satb1 deficient mouse (Satb1^{f1/f1}ThpokCre⁺) or the wild type mouse (Satb1^{f1/f1}).

FIG. 15 illustrates results of FACS analysis on Foxp3 and CD25 of CD4⁺ T cells of mesenteric lymph nodes and spleens of enteritis model mice on day 53 after the transplantation of CD4⁺CD25⁻CD45RB^{hi} T cells isolated from the periphery of the Satb1 deficient mouse (Satb1^{f1/f1}ThpokCre⁺) or the wild type mouse (Satb1^{f1/f1}).

FIG. 16 illustrates results of FACS analysis on IL-17A and IFNg of CD4⁺ T cells of mesenteric lymph nodes and spleens of enteritis model mice on day 53 after the transplantation of CD4⁺CD25⁻CD45RB^{hi} T cells isolated from the peripheries of the Satb1 deficient mouse (Satb1^{f1/f1}ThpokCre⁺) or the wild type mouse (Satb1^{f1/f1}).

FIG. 17 provides graphs plotting the results illustrated in FIGS. 15 and 16.

FIG. 18 illustrates the amount of Foxp3 induced in CD8-positive cells of a T cell specific Satb1 deficient mouse observed by means of FACS.

FIG. 19 illustrates the ratio of Foxp3 expressing cells in the CD8-positive cells.

FIG. 20 illustrates the amount of Foxp3 induction in SATB1 deficient T cells observed by means of FACS. The ratio of Foxp3 expressing cells in Naive T cells, CD25− effector T cells and CD25+ effector T cells after being stimulated by a T cell activator.

FIG. 21 illustrates the results of luciferase analysis of Foxp3 transcriptional activity in Satb1 deficient mouse EL4 cells induced by means of the CRISPR/CAS9 system.

### DESCRIPTION OF EMBODIMENT

According to the present application, expression of Satb1 or the function of Satb1 is inhibited in peripheral conventional T cells. In this specification and claims, the term “peripheral conventional T cells” refers to T cells except for regulatory T cells present in the periphery, and may comprise CD4⁺ cells and CD8⁺ cells. In this application, the cells in which the expression or the function of Satb1 is suppressed may preferably be CD4⁺CD25⁻ cells, for example, CD4⁺CD25⁻CD45RA+cells (naive Th cells), CD4⁺CD25⁻/lowCD45RA⁻ cells (effector Th cells), and CD8+ cells.

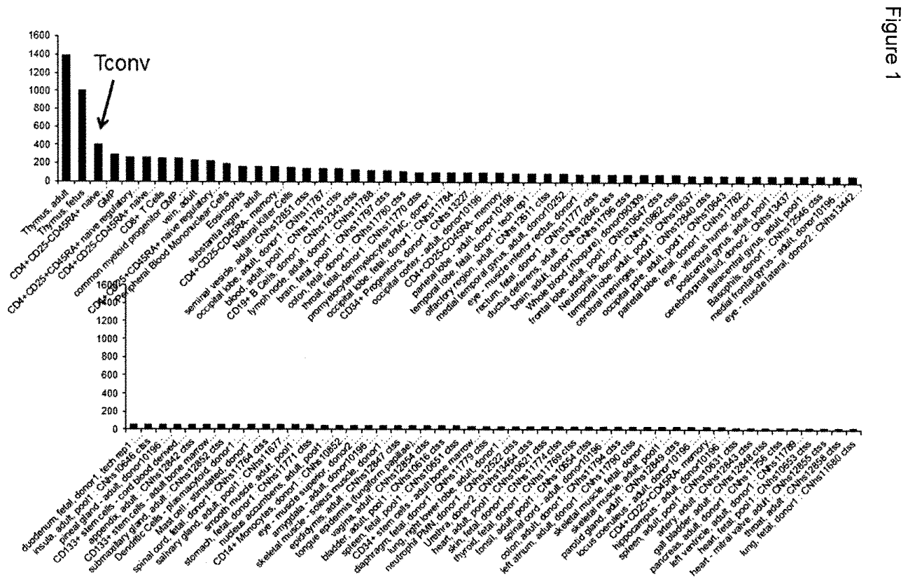

Satb1 (Special AT-rich binding protein 1) is a genome organizer that controls the chromatin structure and the gene expression while collecting a large number of genes to Satb1 and interacting with various enzymes involved in chromatin remodeling. The expression of Satb1 was studied in 293 types of human cells, and the top 100 cells having high-level expression are illustrated in FIG. 1. Satb1 expresses at the highest level in thymocytes, and is regarded to be involved in the generation of thymus in embryos or neonates. On the other hand, in an adult in which thymus has regressed, Satb1 is presumed to express at the highest level in the naive T cells.

In the specification and claims, the expression “CD4 positive” or “CD4⁺” refers to “CD4-positive CD8-negative cells” i.e. “CD4 single positive cells” unless otherwise indicated. The expression “CD8 positive” or “CD8+” refers to “CD4-negative CD8-positive cells” i.e. “CD8 single positive cells” unless otherwise indicated.

A technique to inhibit the expression of Satb1 is not especially limited, and any of known techniques to control gene expression no matter whether it is performed in vivo or in vitro may be employed. Examples include a method for inhibiting the expression using an RNA molecule such as siRNA, shRNA, miRNA, stRNA or antisense RNA, and a method for inhibiting the expression by employing a genome editing technology such as CRISPR/Cas9 or TALEN. Genome editing technologies such as CRISPR/Cas9 are preferably used to inhibit the expression of Satb1.

Examples of methods for inhibiting the function of Satb1 may include, but are not limited to, contacting peripheral T cells with a neutralizing antibody against SATB1 protein or a fragment of the antibody, or a substance such as a small molecular substance that inhibits the activity of SATE1 protein.

In one aspect of the present application, a method for inducing regulatory T cells in a living body, including administering to the subject a substance that inhibits expression of Satb1 or inhibits the function of Satb1 is provided. The present application also provides an inducer for regulatory T cells containing a substance that inhibits the expression of Satb1 or inhibits the function of Satb1.

In another aspect, a method including the step of inducing expression of Foxp3 in vitro by inhibiting expression of Satb1 in peripheral conventional T cells.

Peripheral conventional T cells can be obtained by fractionating the peripheral blood mononuclear cells of an animal including a human to give the CD4⁺or CD8+ fraction, preferably, CD4⁺CD25⁻ fraction or CD8+ fraction by means of, for example, FACSAria (BD Bioscience). Peripheral conventional T cells having a desired antigen specificity may be selected or induced from peripheral conventional T cells. Alternatively, peripheral conventional T cells may be conventional T cells induced from pluripotent stem cells of an animal including a human, such as ES cells and iPS cells. Examples of the procedures for inducing T cells from pluripotent stem cells may include those disclosed in WO 2016/010148, WO 2016/010153, WO 2016/010154, WO 2016/010155, WO 2017/159087, WO 2017/159088 and WO 2017/179720. For inhibiting the Satb1 expression in vitro, any of the above-described known methods may be employed.

According to the method including the step of inducing expression of Foxp3 in vitro may comprise the step of culturing the cells with suppressed Satb1 expression in the presence of a T cell activator. The T cell activator is a substance that induce proliferation and activation of T cells in vitro. Examples may include cytokines such as IL-2, anti-CD3 antibody and anti-CD28 antibody. In addition, the T cell activator may be used in combination with TGFP. By incubating the cells with suppressed Satb1 expression in the presence of the T cell activator, cells expressing Foxp3 specifically proliferate.

When the expression of Satb1 in peripheral conventional T cells is inhibited in vivo or in vitro, the cells express Foxp3, the master transcription factor of regulatory T cells. CD4⁺CD25⁻ cells expressing Foxp3 are facilitated to differentiate into stable Foxp3⁺CD4⁺CD25⁺ cells in the living body and function as regulatory T cells. CD8⁺ cells expressing Foxp3 also differentiate into cells that function as regulatory T cells.

The present application further provides a method for inducing regulatory T cells in a subject, including transferring Foxp3 expressing cells having been induced in vitro from peripheral conventional T cells into the subject.

According to the present application, Foxp3-expressing cells with degree of demethylation in the CNS2 region of Foxp3 gene is 50% or more, preferably 80% or more, and more preferably 90% or more can be obtained.

According to the present application, by inducing regulatory T cells in the living body, diseases and symptoms in which immune tolerance is required to induce, such as autoimmune diseases, allergic diseases, rejection occurring in organ transplantation, graft versus host disease (GVHD) occurring after bone marrow transplantation, and immune metabolic diseases can be treated, or the maternal-fetal immune tolerance can be induced. Accordingly, the method of the present application is useful for the treatment of these diseases and symptoms.

In the specification and claims, the term “treatment” embraces all management of diseases or symptoms including prevention of a disease or a symptom, cure, symptomatic relief, symptom reduction and progression inhibition.

By the method provided herein, Foxp3 expression can be induced not only in the CD4 positive T cells but also in the CD8 positive T cells. CD8 positive T cells recognize Class I major histocompatibility complex (MHC) expressed on the antigen presenting cells and an antigen peptide bound to the MHC molecule. On the other hand, CD4 positive T cells recognize Class II MHC and antigen peptide bound to the MHC molecule. That is, CD4 positive T cells and CD8 positive T cells recognize different antigen and the method provided herein can achieve antigen specific immune suppression for wide variety of immune responses.

Foxp3 positive cells induced in vitro may be dispersed in an appropriate medium and administered to a subject. Examples of the medium for dispersing the cells therein include saline and PBS. The cells may be administered intravenously to a patient. A dosage, the number of times of administration and administration timing may be appropriately set in accordance with the purpose of the induction of regulatory T cells.

In all the aspects, regulatory T cells are presumed to be induced in a subject when immune tolerance is desired to induce. For example, for treating an autoimmune disease, it is presumed that regulatory T cells need to be persistently induced, and for treating or preventing a seasonal allergic disease such as hay fever, the administration timing may be determined in accordance with the generation of the allergen of interest. For organ transplantation or bone marrow transplantation, it is presumed that regulatory T cells are administered when the immune tolerance is desired to induce, for example, simultaneously with the transplantation or when rejection occurs.

The present application also provides a method for screening a regulatory T cell inducer, including the steps of:
(1) treating cells expressing Satb1 with a substance that is a candidate for a regulatory T cell inducer;
(2) measuring the expression of Satb1 or activity of Satb1 of the cells; and
(3) selecting a substance that inhibits the expression of Satb1 or the activity of Satb1, as a regulatory T cell inducer.

The regulatory T cell inducer is useful for the prevention or the treatment of autoimmune diseases, allergic diseases, organ transplantation, bone marrow transplantation, and immune metabolic diseases, or the induction of maternal-fetal immune tolerance.

### EXAMPLES

The present invention is supported by data obtained in the following animal experiments.

### Example 1

### Expression of Foxp3 in CD4⁺ T Cells of Satb1 Deficient Mice

Mouse having a conditional deletion of Satb1 in the T cells was created. Satb1^{f1/f1}ThpokCre⁺ mouse was created by crossing Satb1^{f1/f1} mouse (Hao, B. et al., J. Exp. Med. 212 809-824 (2015)) and ThpokCre⁺ mouse (C57BL/6J) (Mucida D., et al., Nat. Immunol. 14, 281-289 (2013)) created by previously reported procedures. As a control, Satb1^{f1/+}ThpokCre⁺ mouse was created in the same manner. In order to confirm the expression of Foxp3, each mouse was crossed with Foxp3^{GFP} mouse.

In the Satb1^{f1/f1}ThpokCre⁺ mouse, Satb1 deletion was induced in peripheral thymus-derived Treg, conventional T cell (Tconv) and Treg differentiated from Tconv in the periphery (pTreg), while the expression of Satb1 in Treg (tTreg) in the thymus and Tconv in the thymus was not affected. Hereinafter, the Satb1^{f1/f1}ThpokCre⁺ mouse is designated as “Satb1 deficient mouse”, and the Satb1^{f1/f1}ThpokCre⁺ mouse is designated as “control mouse”.

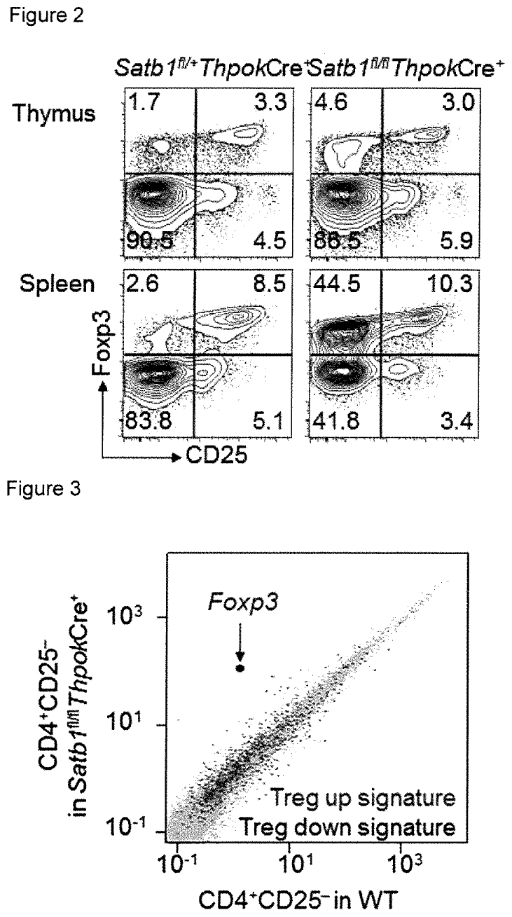

The thymus and spleen of each mouse were taken out to examine the expression of Foxp3 and CD25 in the CD4 positive cells. The results are illustrated in FIG. 2. In the thymus, the expression of Satb1 was not inhibited, and the expression levels of Foxp3 were not significantly different between the Satb1 deficient mouse and the control mouse. In the spleen, about 10% of the CD4 positive cells of the control mouse expressed Foxp3, while .50% or more of the CD4 positive cells of the Stab1 deficient mouse expressed Foxp3.

In peripheral CD4⁺CD25⁻ cells of the Satb1 deficient mouse and of the wild type mouse, gene expression profile was examined. Peripheral CD4⁺CD25⁻ cells of each mouse were sorted by FACS Aria II, and RNAs were extracted from 1×10⁵ cells by using TRIzol Reagent (Thermo Fisher). Thus obtained RNAs were purified using miRNeasy Micro Kit (Quiagen) and a RNA library was prepared by using Ion Total RNA-Seq Kit v2 (Thermo Fisher), and RNA sequences were confirmed by using Ion Proton. The resultant sequences were mapped to the mouse genome (mmm9) by TopHat2 (v. 2.0.11). On the basis of the results of mapping, normalized expression level FPKMs were calculated by Cuffnorm (v. 2.2.0) and differentially expressed genes (FDR<0.05) were identified by Cuffdiff (v. 2.2.0). In peripheral regulatory T cells (Treg), genes expressed at higher levels (Treg up signature gene) and genes expressed at lower levels (Treg down signature) than those in peripheral conventional T cells (Tconv) were identified and respectively plotted. The results are illustrated in FIG. 3.

Among genes expressed at higher levels in Treg, the expression of Foxp3 was remarkably increased in the Satb1 deficient mouse. The expression levels of the genes expressed at higher levels in peripheral Treg cells, namely, CD25, GITR, CTLA4, Helios and Nrp1, were not different between peripheral Foxp3⁺CD4′CO25⁺ cells derived from the control mouse and those derived from the Stab1 deficient mouse.

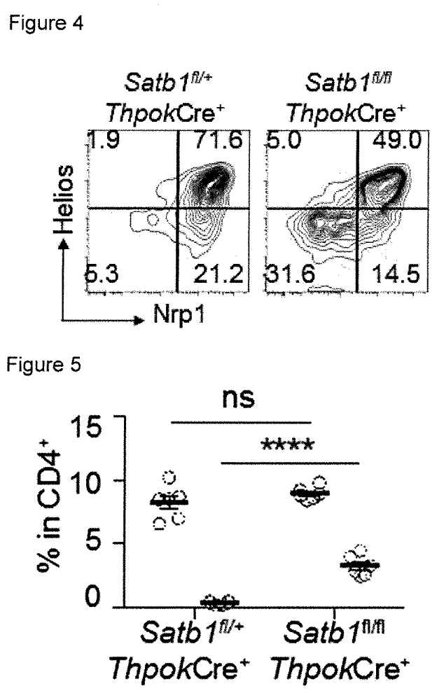

The expressions of Helios and Nrp1 in peripheral CD4⁺CD25⁺Foxp3⁺ T cells or regulatory T cells obtained from the Satb1 deficient mouse and the control mouse were confirmed by FACS. Nrp1⁻Helios⁻Foxp3⁺ cells are regulatory T cells derived from the periphery and Nrp1⁺Helios⁺Foxp3⁺ cells are regulatory T cells derived from the thymus. The results are illustrated in FIGS. 4 and 5. In FIG. 5, with respect to the control mouse (Satb1^{f1/+}ThpokCre⁺) and the Satb1 deficient mouse (Satb1^{f1/f1}ThpokCre⁺), the ratio of Nrp1⁺Helios⁺ Treg cells or regulatory T cells derived from the thymus is shown on the left side, and the ratio of the Nrp1⁺Helios⁻ Treg cells or regulatory T cells in the CD4-positive cells derived from the periphery is shown on the right side. It was confirmed that peripherally derived regulatory T cells increased in the Satb1 deficient mice. That is to say, when the expression of Satb1 is inhibited, Foxp3 induction will be observed in the periphery.

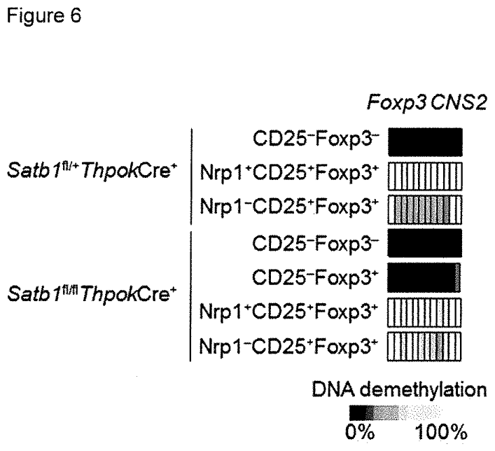

Stable expression of Foxp3 results from demethylation of Foxp3 gene. Therefore, DNA demethylation in the CNS2 region of Foxp3 gene in each cell fraction was examined. The results are illustrated in FIG. 6. In regulatory T cells (NrP1⁻CD25⁺Foxp3⁺) derived from the periphery of the Satb1 deficient mouse, 30% or more DNA demethylation was observed. This degree of demethylation was substantially the same as in regulatory T cells derived from the periphery of the control mouse. Based on this result, it was confirmed that the periphery derived regulatory T cells induced in the Satb1 deficient mouse stably expressed Foxp3 by means of DNA demethylation of Foxp3 gene.

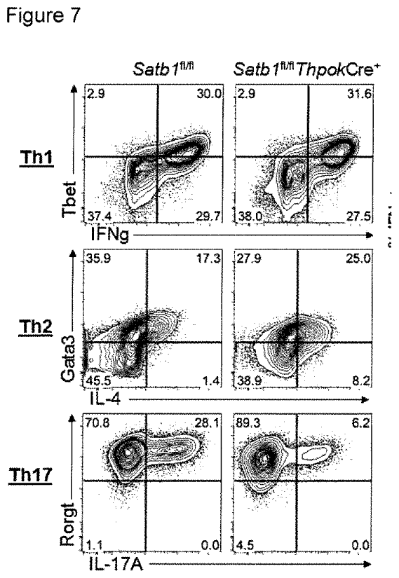

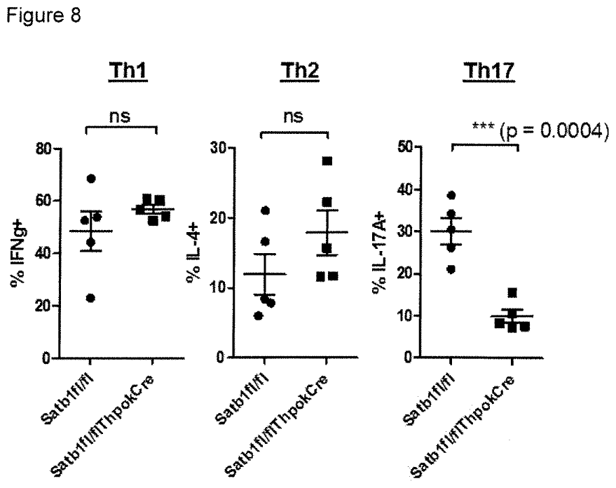

Peripheral CD4⁺CD25⁻CD44⁻ cells of the wild type mouse Satb1^{f1/f1}) and those of the Satb1 deficient mouse (Satb1^{f1/f1}ThpokCre⁺) were obtained and cultured under the conditions for inducing Th1, Th2 and Th17 cells respectively. In the thus obtained cells, Th1, Th2 and Th17 cells were detected by flow cytometry using markers for these cells. The results are illustrated in FIGS. 7 and 8. Between the Satb1 deficient mouse and the wild type mouse, there was no significant difference in the amount of the Th1 and Th2 cells, but the differentiation into the Th17 cells that produce inflammatory cytokines was inhibited. Therefore, when the expression or the function of Satb1 in the peripheral conventional T cells is inhibited, not only the induction of immune tolerance through the induction of regulatory T cells but also inhibition of inflammation through the decrease of the Th17 cells are expected.

### Example 2

### Transplantation of Satb1 Deficient T Cells in the Living Body

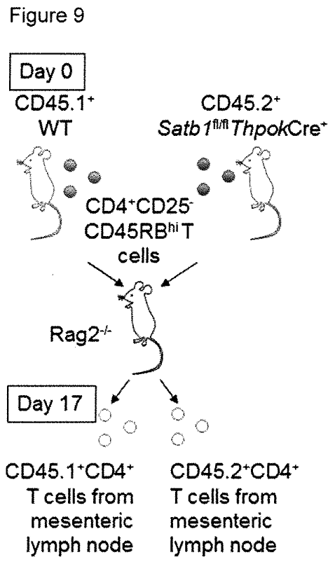

The outlines of this study are illustrated in FIG. 9. CD4⁺CD25⁻CD45RB^{hi} T cells were sorted from the lymphocytes of a CD45.1⁺ wild type mouse and a CD45.2⁺ Satb1 deficient mouse (Satb1^{f1/f1}ThpokCre⁺) by FACS. A mixture of 2.5×10⁵ cells each of these cells was transferred into a Rag2^{-/-} mice by intravenous administration. The Rag2^{-/-} mouse is an immunodeficient mouse that cannot reconstitute T cell and B cell receptors and hence is completely deficient in T cells, B cells and NKT (natural killer T) cells.

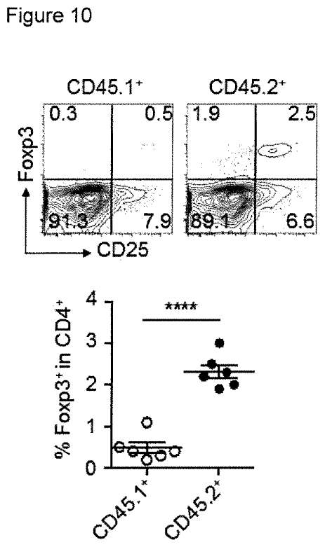

On day 17 after the transplantation, lymphocytes were isolated from the mesenteric lymph node and analyzed by FACS. Results obtained from 6 animals are shown in the graph illustrated in the lower portion of FIG. 10. As compared with the CD45.1⁺CD4⁺ cells derived from the wild type mice, the expression of Foxp3 was remarkably increased in the CD45.2⁺CD4⁺ cells derived from the Satb1 deficient mice.

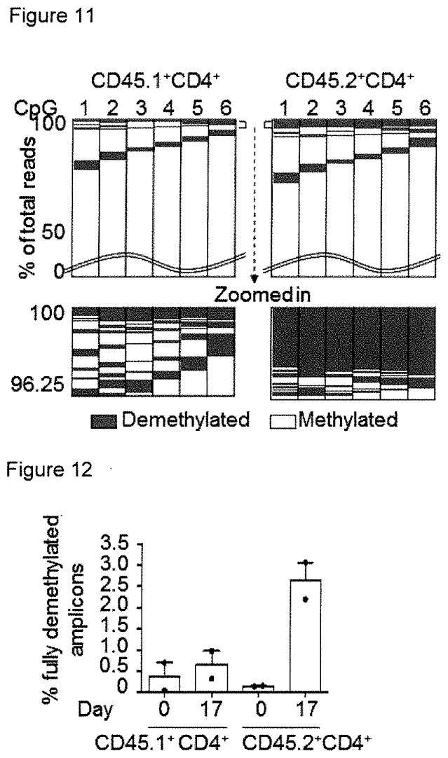

Besides, the degrees of methylation of six CpG residues present in the CNS2 region of Foxp3 gene expressed in each of the CD45.1⁺CD4⁺ T cells and the CD45.2⁺CD4⁺ T cells obtained from the mesenteric lymph nodes of the mice on day 17 after the transplantation were observed. The results are illustrated in FIG. 11. The CpG residues were numbered as 1 to 6 successively from the 5′ end (shown as columns). Respective amplicons were aligned successively from the most demethylated residue (shown as rows). The full demethylation is taken as 100% and the portion corresponding to 0-50% demethylation was omitted from the figure (corresponding to the portion below a wavy line). Clones corresponding to top 3.75% from the most demethylated one are enlarged in the lower portion of the figure. FIG. 12 shows ratios of fully demethylated amplicons on day 0 and day 17 after the transplantation. In the Satb1 deficient CD4⁺ T cells, the demethylation of Foxp3 gene in the CNS2 region proceeded and the gene stably expressed in the cells.

### Example 3

### Effect of In Vivo Transplantation of Satb1 Deficient T Cells into Enteritis Model Mice

Four-weeks-old Rag2^{-/-} mice were used as enteritis model mice (Powrie et al., 1993, Int Immunology).

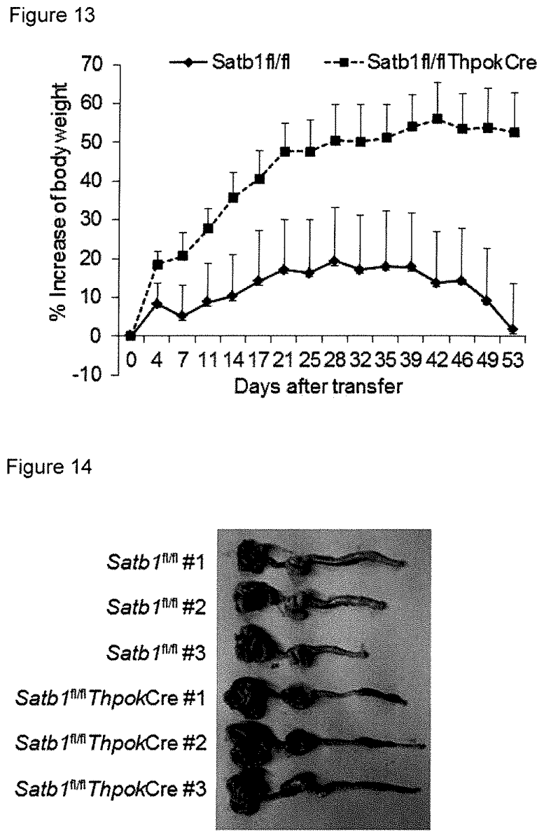

CD4⁺CD25⁻CD45RB^{hi} T cells were obtained by sorting lymphocytes of the wild type mouse (Satb1^{f1/f1}) and the Satb1 deficient mouse (Satb1^{f1/f1}ThpokCre⁺) by FACS. The cells (1×10⁶ cells) of each mouse were transplanted into a 4-week-old Rag2^{-/-} mouse by intravenous administration. Changes in body weights after the cell transplantation were observed up to day 53. On day 53 after the transplantation, the animals were euthanized and their colons were obtained. The CD4⁺CD25⁻CD45RB^{hi} T cells taken out from three Satb1 deficient mice and three wild type mice were transplanted respectively into different Rag2^{-/-} mice. The changes in the body weights are illustrated in FIG. 13, and the photographs of the colons are illustrated in FIG. 14.

In the mice into which the CD4⁺CD25⁻ T cells derived from the Satb1 deficient mouse had been transplanted, the body weights increased significantly as compared with the mice into which the wild type T cells had been transplanted. On day 53 after the transplantation, the colons removed from the mice were visually observed and confirmed that the colon removed from the mice transplanted with the T cells of Stab1 deficient mouse were longer than those removed from the mice transplanted with the wild type T cells. It was confirmed that the onset of enteritis was inhibited by the transplantation of the T cells from the Satb1 deficient mouse.

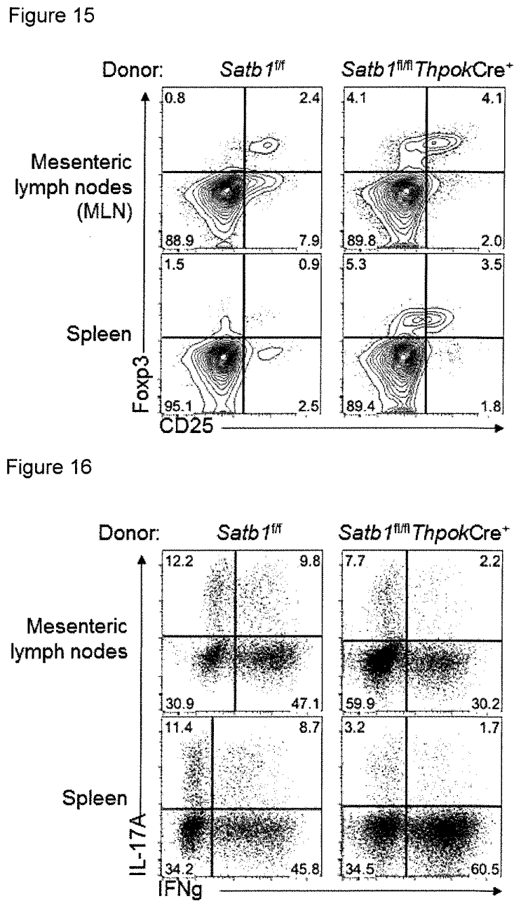

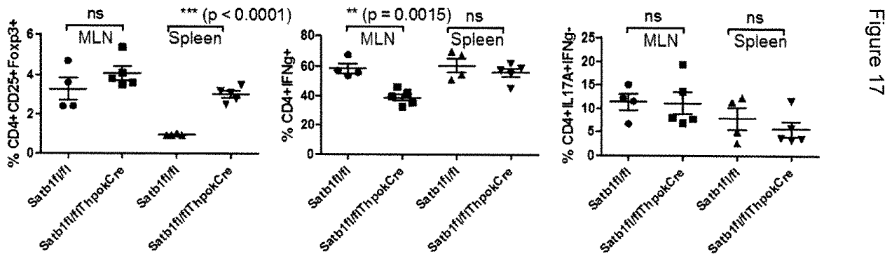

On day 53 after the transplantation, CD4⁺ T cells were collected from the mesenteric lymph node and spleen of the enteritis model mice (recipient) into which T cells of the wild type mouse or the Satb1 deficient mouse (donor) had been transplanted and the cells were analyzed by FACS. The results are illustrated in FIGS. 15 and 16. FIG. 17 provide a graph summarizing those results.

In the enteritis model mice into which T cells of the Satb1 deficient mouse had been transplanted, the number of the cells expressing Foxp3 increased and the number of regulatory T cells or Foxp3⁺CD4⁺CD25⁺ cells increased as compared with those in the mice transplanted with the T cells from the wild type mouse. On the other hand, in the mesenteric lymph node, the number of the Th1 cells decreased significantly while the number of the Th17 cells did not change substantially.

### Example 4

### Induction of Foxp3 Expression in CD8-Positive T Cells from Satb1-Deficient Mouse

A mouse having a conditional deletion of Satb1 in the CD4 positive T cells was created. Satb1^{f1/f1} mouse (Hao, B. Et al., J. Exp. Med. 212 809 -824 (2015)) prepared by the previously reported method was crossed with CD4Cre⁺ mouse (C 57BL/6J) (Lee, PP et al., Immunity 15. 763 -774 (2001)) to obtain Satb1^{f1/f1}CD4Cre⁺ mouse. CD4⁻Cre⁺ mouse was used as control.

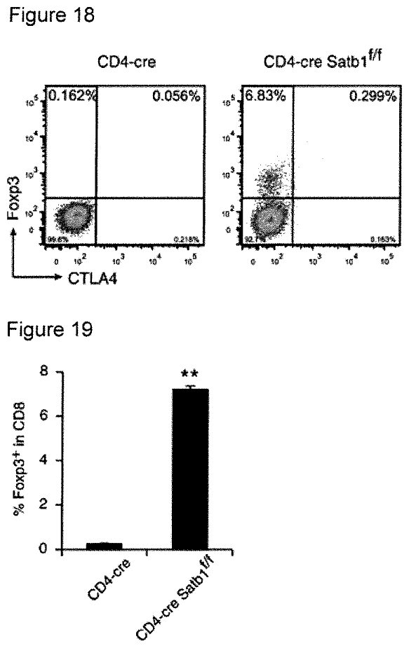

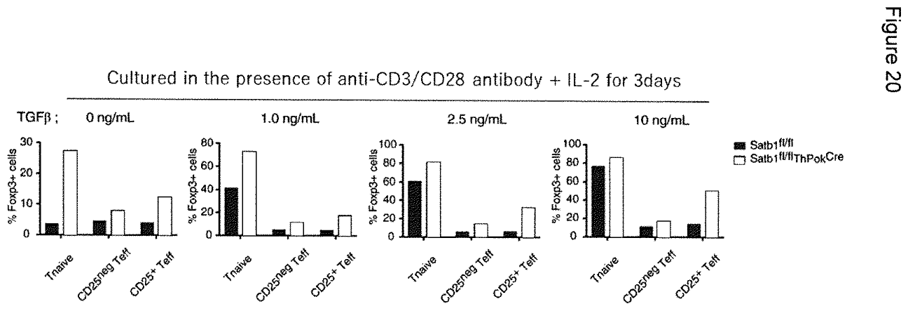

The lymph nodes were collected separately from both mice, the tissues were disrupted with frosted glass, and filtered through nylon mesh to prepare a whole lymphocyte cell suspension. The prepared whole lymphocyte cells were stained with anti-CD4 antibody, anti-CD8 antibody, anti-Foxp3 antibody and anti-CTLA4 antibody, and Foxp3 and CTLA4 expression in CD8-positive cells were analyzed using FACSAria II. Results of FACS analysis are shown in FIG. 18, and the percentage of Foxp3-expressing cells among CD8-positive cells is shown in FIG. 19.

Because all T cells in the thymus differentiate into the respective SP cells through the CD4⁺CD8⁺ DP state, CD8-positive cells from the Satb1^{f1/f1}CD4Cre⁺ mouse lack Satb1. Since the induction of Foxp3 by the deficiency of Satb1 occurs in the periphery, this example shows that it is possible to induce Foxp3 expressing T cells from CD8-positive T cells by inhibiting Satb1.

### Example 5

### Induction of Foxp3 Expression in the Effector T Cells from Satb1-Deficient Mice

The lymph nodes of Foxp3-DTR-GFP KI/ThPOK-Cre/SATB1^{f1/f1} mice obtained in the same manner as in Example 1 were collected. The tissues were crushed using frosted glass, and filtered with nylon mesh to prepare a whole lymphocyte cell suspension. The prepared whole lymphocyte cells were stained with anti-CD4 antibody, anti-CD25 antibody, anti-CD44 antibody, and anti-CD62L antibody, and each of the cell fractions was purified using FACSAria II. Naïve Th cells (CD25⁻CD44⁻CD62L⁺), CD25⁻ effector (CD25⁻GFP⁻CD44⁺CD62L⁻) and CD25⁺ effector (CD25⁺GFP⁻CD44⁺CD62L⁻) cells were cultured with 1-10 ng/mL TGF in the presence of Dynabeads® T cell activator (anti-CD3/CD28 antibody) (Thermo Fisher Scientific, Inc.) and IL-2 for 72 hours. The proportion of GFP-positive cells in the stimulated cells was analyzed by FACS. The results are shown in FIG. 20.

The expression of Foxp3 in the CD4⁺CD25⁺ effector cells deficient in Satb1 was enhanced by stimulating the cells in vitro with a T-cell activator.

### Example 6

### Promotion of Foxp3 Expression by Inducing in Vitro Satb1 Inhibition Using the CRISPR/CAS System

A SATB1-deficient EL4 cell line was produced by using the CRISPR/CAS9 system from a mouse T lymphoma cell line, EL4 cells. The guide RNA was incorporated into pSpCas9-T2A-GFP/sgRNA. The original vector pSpCas9n (BB)-2A-GFP(PX 461), Addgene plasmid #48140 was gifted from Professor Feng Zhang. The guide sequence used here was sgSatb1:

caccgCGCCGGGCGGCGGACTTCCC

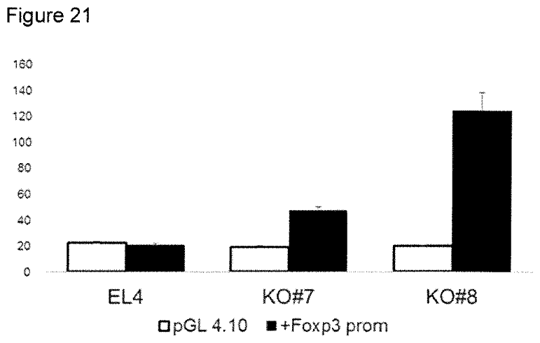

Cas9 and sgRNA expression vector was introduced into the cells using Nucleofector L system (Lonza). Luciferase expression vector containing the Foxp3 promoter sequence was introduced into the generated cells by Nucleofector L system and stimulated by Dynabeads (R) T cell activator for 24 hours. Cells were lysed after the stimulation and Luciferase activity was measured. The results are shown in FIG. 21. EL4 shows cells in which Satb1 was not knocked out, and KO #7 and KO #9 show the cells in which Satb1 was knocked out. pGL 4.1 represents a negative control that did not contain the Foxp3 promoter sequence.

It was confirmed that the expression of Foxp3 could be induced in vitro by inhibiting the expression of Satb1 in T cells.

### INDUSTRIAL APPLICABILITY

According to the method provided herein, the induction of stable regulatory T cells have become possible. By inducing the stable regulatory T cells, the treatment of diseases or conditions such as autoimmune diseases, allergic diseases, rejections occurring in organ transplantation or graft versus host disease occurring in organ transplantation, or the induction of maternal-fetal immune tolerance can be achieved.

What is claimed is:

1. A method for inducing or generating regulatory T cells, comprising the step of inhibiting the expression of Satb 1 or the function of Satb 1 in peripheral conventional T cells.

2. The method according to claim 1, comprising administering a substance that inhibits the expression of Satb1 or the function of Satb1 to a subject, thereby inducing regulatory T cells in the subject.

3. The method according to claim 1, the expression of Foxp3 in peripheral conventional T cells is induced by inhibiting expression of Satb1 in the peripheral conventional T cells in vitro.

4. The method according to claim 3, further comprising the step of culturing the cells with suppressed Satb 1 expression in the presence of a T-cell activator.

5. The method according to claim 1, wherein the peripheral conventional T cells are CD4-positive cells or CD8-positive cells.

6. The method according to claim 3, wherein the expression of Satb 1 is inhibited by means of a genome editing technology.

7. A method for inducing regulatory T cells in a subject, which comprises the step of transferring Foxp3 expressing T cells obtained by the method according to claim 3 into the subject.

8. The method according to claim 2, which is for a treatment selected from the group consisting of the treatment of an autoimmune disease, the treatment of an immune metabolic disease, the treatment of an allergy, the treatment of rejection occurring in organ transplantation, the treatment of a graft versus host disease occurring in organ transplantation, and the induction of maternal-fetal immune tolerance.

9. (canceled)

10. An agent comprising a substance that inhibits the expression of Satb1 or the function of Satb1, for use in a treatment selected from the group consisting of the treatment of an autoimmune disease, the treatment of an immune metabolic disease, the treatment of an allergy, the treatment of rejection occurring in organ transplantation, the treatment of a graft versus host disease occurring in organ transplantation, and the induction of maternal-fetal immune tolerance.

11. (canceled)

12. A method for screening a regulatory T cell inducer, comprising the steps of:
(1) treating cells expressing Satb1 with a substance that is a candidate for a regulatory T cell inducer;
(2) measuring the expression or the function of Satb1 in the cells; and
(3) selecting a substance that inhibits the expression or the function of Satb1 as a regulatory T cell inducer.

13. The method according to claim 7, which is for a treatment selected from the group consisting of the treatment of an autoimmune disease, the treatment of an immune metabolic diseases, the treatment of an allergy, the treatment of rejection occurring in organ transplantation, the treatment of a graft versus host disease occurring in organ transplantation, and the induction of maternal-fetal immune tolerance.
